(19) United States
(12) Patent Application Publication
(10) Pub. No.: US 2017/0095631 A1
(43) Pub. Date: Apr. 6, 2017

(54) RESUSCITATOR WITH DISTAL OXYGEN INLET, BREATHING CIRCUITS HAVING REUSABLE AND DISPOSABLE COMPONENTS, SYSTEMS AND METHODS FOR RESUSCITATION AND PROVIDING ASSISTED VENTILATION AND ANESTHESIA, AND KITS AND COMPONENTS THEREFORE

(71) Applicant: Atsuo F. Fukunaga, Palos Verdes Peninsula, CA (US)

(72) Inventor: Atsuo F. Fukunaga, Palos Verdes Peninsula, CA (US)

(21) Appl. No.: 15/282,565

(22) Filed: Sep. 30, 2016

Related U.S. Application Data
(60) Provisional application No. 62/236,121, filed on Oct. 1, 2015.

(51) Int. Cl. A61M 16/08; A61M 16/06; A61M 16/01; A61M 16/12; A61M 16/20; A61M 16/10; A61M 16/00; A61M 16/04

(52) U.S. Cl. CPC A61M 16/0875; A61M 16/0078; A61M 16/0666; A61M 16/04; A61M 16/125; A61M 16/208; A61M 16/0084; A61M 16/1065; A61M 16/107; A61M 16/01; A61M 2205/75

(57) ABSTRACT

A resuscitation device is disclosed comprising a bag, a non-rebreathing valve (NRV), a breathing conduit, a filter, a pressure limiting valve, a patient airway device, and an oxygen inlet located distally (on the patient side) of the bag. The device can be operatively connected to a patient airway device to provide gases to and/or exhaust gases from a patient. Also disclosed are methods of using the resuscitation device, components, and kits for constructing same. Breathing circuits for use in assisted ventilation and anesthesia are disclosed having disposable and reusable circuit components along with systems incorporating same. Methods of using the breathing circuits are described, along with descriptions of circuit components and kits for constructing systems using the breathing circuits.

### FIELD OF THE INVENTION

The present invention relates to devices or apparatus for use in resuscitating and/or providing assisted ventilation or anesthesia to patients in a variety of settings, such as in operating rooms, intensive care units, emergency medicine clinics, ambulances, and trauma situations. The present inventions also relate to systems and methods for connecting patients to anesthesia machines, ventilators, manual resuscitators and the like. More particularly, the inventions relate to filters and breathing circuits comprising a disposable component and a reusable component, which leads to a substantial reduction in medical waste, yet provides a multifunctional and versatile respiratory device that has minimal flow resistance and adjustable apparatus dead space.

### BACKGROUND OF THE INVENTION

The primary goal in ventilation is to maintain oxygenation to avoid hypoxic brain injury, or death. Manual ventilation is commonly used in emergencies. Patients in need of emergent ventilation require immediate availability of air and or supplemental oxygen.

RESUSCITATORS. Manual ventilation can be provided by pumps, which are usually self-inflating bags formed of an elastomeric material. Such bags are operated by squeezing (compressing) and releasing (decompressing) the bag; for example, when the bag is compressed air is forced out of an outlet via a first one-way valve, and when the bag is released, air from the surrounding atmosphere enters the bag via a second one-way valve. For ease of description, in methods, systems and components for providing assisted ventilation, parts that would in use be aimed towards a patient or located closest to a patient are referred to as distal, and parts that would in use be aimed away from a patient or located furthest away from a patient are known as proximal. So, for the bag described above, the air outlet would be at the distal end of the bag and the air inlet would be proximal of the air outlet.

A common resuscitation system, referred to as a Bag-Valve-Mask (BVM) resuscitator comprises a bag as described above, a pressure limiting valve (third valve) distal of the bag, and a mask for providing air to a patient. To facilitate gripping and squeezing, the bag is usually oval shaped (resembling a ball used in American football). Air expelled from the bag is carried via a conduit to the mask. Patients are resuscitated by alternately squeezing the bag to expel air from the mask to the patient and thereby provide positive pressure ventilation, followed by releasing the bag to refill the bag while the patient expires. An oxygen (O2) inlet and reservoir are located proximally to the bag; O2 is supplied via the O2 inlet to the reservoir. The main concept, structure and/or configuration of the BVM (e.g., Ambu® Bag) has not changed since it was first developed in 1953.

An important thing to consider is that medical patients experiencing difficulty breathing are usually provided oxygen enriched air (i.e., higher than atmospheric FiO2). The standard FiO2 is typically provided between 0.30 and 0.50. It is known that when a patient wears a nasal cannula or a face mask, each additional liter of oxygen provided adds about 4% to their FiO2. Calculation of the additional O2 flow to the air supplied by a resuscitator to achieve a clinically satisfactory range of FiO2 to a patient in need of oxygenation, ventilation or resuscitation is shown in Table 1 (FIG. 1).

Yet, prior art systems use many times the amount of oxygen calculated in Table 1 because the design and construction of the systems leads to dilution and wasting of O2. In fact, prior art BVMs manufacturers recommend high O2 flows, usually 12 to 15 liter per minute (LPM).

To provide and maintain such high flows of oxygen with prior art systems, large tanks of oxygen must be available and/or an oxygen production means must be available. Providing sufficient oxygen requires significant effort and costs, for example inter alia: (1) human labor to obtain, fill and replace oxygen tanks and/or to make oxygen production systems and associated supplies, (2) storage space for oxygen tanks or production systems in hospital environments is utilized, and hospital space is particularly expensive, (3) in order to have an adequate oxygen supply warehouse space is needed, (4) for emergency oxygen supply, oxygen tanks take up space in ambulances while increasing the use of fuel, and (5) in remote locations it is difficult or impractical to provide large oxygen sources.

Despite the foregoing problems, oxygen utilization efficiency has not been met. In fact, suppliers of BVM resuscitators and health professionals continue to recommend and/or require high O2 inflows, more than 12 to 15 LPM (Liters Per Minute). The O2 is supplied to the reservoir at the proximal end of a resuscitator or assisted ventilation systems, where it is mixed with air from the surroundings. These high flows of O2 limit the usage to a short period of time and/or increase the cost of providing assisted ventilation. Thus, there is a need to reduce oxygen waste when providing assisted ventilation.

### Assisted Ventilation and Anesthesia

Patients in clinical settings are usually provided assisted ventilation by assisted ventilation machines and systems, which use tubes to carry inspiratory gases from a machine to a patient and carry exhaust gases to an expiratory outlet on the machine. Examples of such systems, their components and methods of use are described in U.S. Patent Application Publication US 2005/0188990 A1.

For decades, typical adult circle breathing circuits (for anesthesia use) and ventilator circuits (for use in e.g., an Intensive Care Unit, “ICU”) have been and are still provided in standard lengths of 60, 72 and 108 inches. The use of expandable, pleated tubes (e.g., “flexitube” or “flextube” such as the commercially available Ultra-Flex® by King Systems) provides for a greater range of breathing circuit dimensions, but such adjustable tubes are usually made to conform to the above lengths when expanded, and the entire circuit is disposed of after a single use. Information and specifications on the above circuits can be found in product information associated with devices sold by companies such as Intersurgical, Inc. (of England), Portex, Inc. (of New Hampshire, USA), Westmed Inc. (of Arizona, USA) among many others. Recently, a unilimb breathing circuit comprising the inner and outer tubes formed of pleated tubes has been available in 72 inch and 108 inch lengths when expanded (King Flex2™ Breathing Circuit, Ambu/King Systems Corporation of Indiana, USA).

Standard corrugated tubing, unlike flexitube, cannot be axially extended or compressed to a new self-maintained length. The entire bulky and expensive circuit comprised of tubing and other kit components forming breathing circuits in assisted ventilation systems and methods are disposed of after use.

### Safety has a High Cost in Materials and Pollution

The safety of patients is the foremost concern of healthcare practitioners. The role of respiratory equipment as a source of cross infection leading to respiratory diseases is well known. With the increasing threat of infectious diseases, such as Ebola, SARS, hepatitis, tuberculosis, and HIV, the need to protect respiratory equipment to minimize exposure of patients to infectious respiratory secretions is more compelling than ever. Disposable devices, including breathing circuits and filters, have been widely used to reduce the chance of passing infectious agents between patients. However, the large and ever increasing amounts of medical waste pose serious problems, such as potential toxic environmental effects caused by its disposal and the costs of providing the disposable components. To the extent contaminated equipment can be sterilized for reuse, there are associated high costs for labor, equipment, cleaning supplies, and storage.

Therefore, there is a need for assisted ventilation systems that protect the patient from cross-contamination, yet reduce medical waste and/or the amount of components that are used for a single use before disposal or sterilization. To overcome the problem of cross-contamination, a filter at the distal end of the circuit has been used, but this blocks the facial area and in addition produces dead space by the filter; furthermore, the weight of the filter pulls on and/or moves the airway devices (e.g., endotracheal tube, laryngeal mask, etc.) connected to the patient, which may cause harm to the patient's airway passage and is very inconvenient. To overcome these later problems, a catheter mount consisting of a flexible tube and a filter is being used. However, the catheter mount adds more dead space and cost. An alternative is the use of filters at the proximal end of the conduits (tubes), which is probably the most common use thereof. However, the whole conduits (tubes, circuits) need to be disposed of along with the filters.

The large number of patients using disposable breathing circuits generates a tremendous amount of medical waste. Hauling and disposing of medical waste, particularly transport and disposal outside of urban areas is very expensive. Therefore, there is a compelling need to minimize the amount of plastic and other materials used and disposed of while protecting patients from cross-infection. There is also a need for simple, efficient and convenient resuscitation and assisted ventilation devices that serve multiple functions, yet protect the patient as well as or better than prior art devices, while being more economical to use and minimize disposable material.

The foregoing problems with Resuscitators and Assisted Ventilation Circuits are solved by the present inventions, which are described in more detail below, along with reference to the accompanying Figures, which are first described below.

### DESCRIPTION OF THE FIGURES

FIG. 1 shows Table 1, discussed above, which includes calculations for the oxygen flows needed to achieve higher oxygen content in the air flow inspired by a patient, FiO2.

FIG. 2 shows the conventional, standard components and structure of the presently used BVM resuscitator devices (Bag Valve Mask). A flexible tube is sometimes used instead of the reservoir bag. Notice that the complex structure, bulky bag and valves are at the patient's facial area. The BVM illustrated requires at least 5 valves [patient valve or NRV (non-rebreathing valve), pressure limiting valve or PLV, one-way valve at the proximal end of the Bag, the air-in valve, and the excess 02 valve]. Most importantly, the supplemental 02 is not provided directly to the patient or breathing conduit but directed to a reservoir (in most instances, the 02 inlet is at the proximal end of the bag). Note that this configuration requires very high 02 flows, but most of the supplied oxygen is wasted. Furthermore, to ventilate the patient with the conventional (prior art) resuscitator, the big and bulky bag is close to the face of the patient.

FIG. 3a-1 through 3b-3 illustrate several embodiments of preferred embodiments of the present inventions, which include configurations of new resuscitators of the present inventions (e.g., BVA Tube™ resuscitator). In contrast to the conventional (prior art) resuscitators, there is a breathing conduit 2 (or airway) and filter 3 (such as the EcoFlex Dispo shown in FIGS. 6 and 6a) that provides more space around the facial area of the patient. Conduit 2 and bag 8 are in fluid communication. Moreover, the fresh gases e.g., oxygen (O2) are directly provided to the patient via conduit 2. The non-rebreathing valve 4, O2 inlet 5 and other devices (e.g. pressure limiting valve 6, PEEP 7, manometer etc.) are located, in most instances, at the distal end of the Bag 8 and distal to the first outlet 8a and proximal to breathing conduit 2 and filter 3. It is noted that providing swivel means to the connector fitting 2A at the distal end of the tubing 2 or elbow 900 (if present), is greatly recommended as it allows easy maneuvering of the new resuscitator/oxygenator and a more efficient seal of the mask.

FIG. 4 illustrates an exemplary new system of the present inventions in use, with the upper figure demonstrating flows during the inspiratory phase and the lower figure demonstrating flows during the expiratory phase during provision of resuscitation efforts and/or assisted ventilation to a mammal (although humans are shown, it is envisioned that a wide range of animals may benefit from the present invention with according accommodations to the airway device). The breathing conduit 2 provides space between the face and the Bag 8; continuously inflowing O2 provided in inlet 5 that can be at low flows goes through the inspiratory tube 2a and inflows directly to the patient's airway via an airway device (or “patient airway device”). Also, some of the O2 flows into the distal end of Bag 8 which is mixed with the air coming from the proximal end of Bag 8. During the inspiratory phase (bag compressing phase), the patient is ventilated with the O2 rich gas stored in Conduit 2 plus the mixed gases (O2 supplied from inlet 5 plus air coming from inlet 9). The lower figure shows the exhaled gases going through the expiratory tube 2b is vented out. In this configuration, the conduit is a multilumen, coaxial tube, which requires a coaxial NRV valve such as the ones in FIG. 3a-1-V, but it can be a monolumen tube.

FIG. 5 illustrates an exemplary resuscitator of the present invention wherein the breathing conduit can be a monolumen conduit, with the upper figure demonstrating flows during the inspiratory phase and the lower figure demonstrating flows during the expiratory phase. In this configuration, the non-rebreathing valve 4 (“NRV”) is at the distal end of the breathing conduit in contrast to being at the proximal end of the multilumen conduit (e.g., coaxial tube in FIG. 4). Filter 3, O2 inlet 5, and other components are proximal of the breathing conduit but distal to the Bag. A disadvantage of this configuration is that the NRV 4 adds weight to the conduit and bulkiness at the patient's face. A further limitation of this configuration compared to the multilumen, coaxial conduit configuration shown in FIG. 4 is that it will not readily connect with the EcoFlex Reuse System™ shown in FIGS. 6 and 7.

FIG. 6 shows in its upper portion the layout and components of a commercially available breathing circuit known as the King Universal Flex2 Circuit, which has a patient airway conduit that can be 72 inches long. The patient airway conduit is a multilumen conduit 200, wherein the inspiratory gas tube 200a is contained within the expiratory gas tube 200b, in a unilimb breathing circuit which can connect to a mating multilumen filter (i.e., a filter having separate inspiratory and expiratory chambers) to allow connection to the unilimb inspiratory and expiratory conduits, respectively). In the embodiment shown, the inspiratory conduit is coaxially located within the expiratory conduit, and each conduit is connected, respectively, through a coaxial filter 300, to a machine inspiratory gas source and expiratory gas port via a manifold 1000 (the coaxial filter also serves as a proximal fitting or coupling to distal and proximal components in the breathing circuit connected to the assisted ventilation machine; see the encircled figure to the right that shows the direction of flow of gases, inspired O2 and expired CO2). With respect to fittings, such as the proximal fitting, in preferred embodiments the fittings comprise a more rigid material than the flexible tubes. For example, the fittings may have rigid tubes or pipes formed of a rigid plastic or other material that facilitates sliding friction fit engagement with mating fittings. Thus, the mating fitting would have a pipe or pipes that each has a diameter either slightly larger or smaller than the pipe in the fitting to which sliding friction fit is desired. In some embodiments, one or more flexible tubes may be bonded to the pipe end or pipe ends of a fitting, enable quick connection and disconnection of the flexible tube or tubes to other components of devices and systems of the present inventions. Suitable fitting and flexible tube materials include, but are not limited to, the materials used in the fittings and flexible tubes of the King Universal Flex2 Circuit.

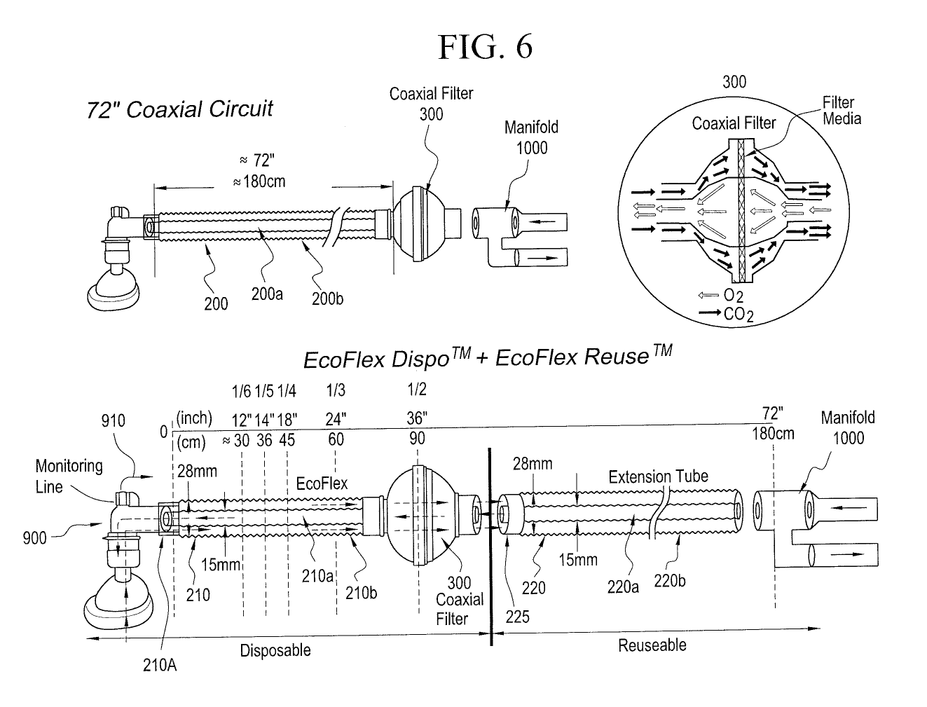

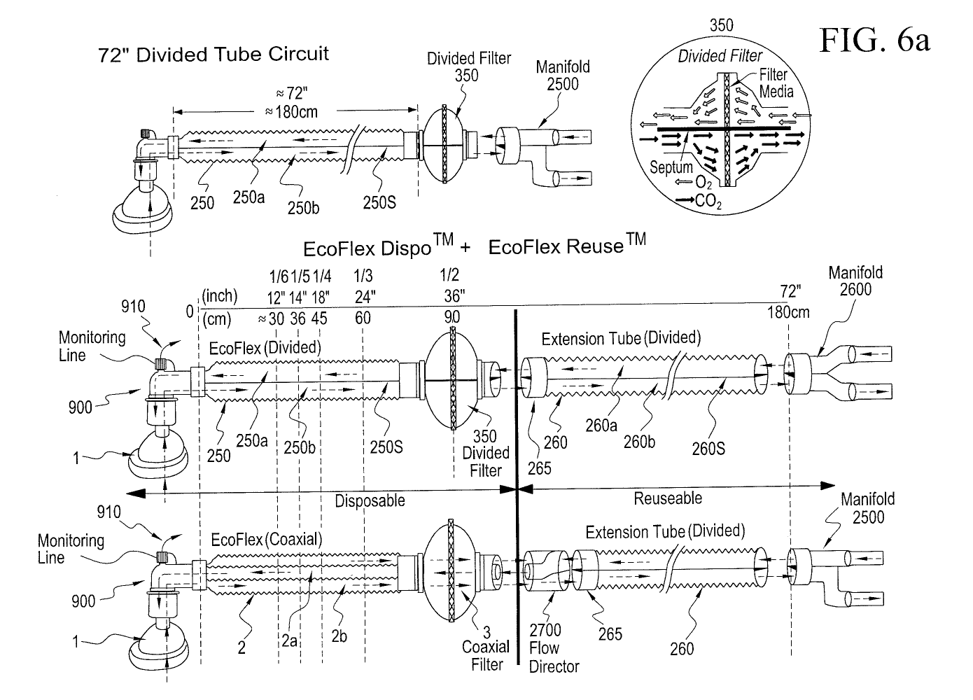

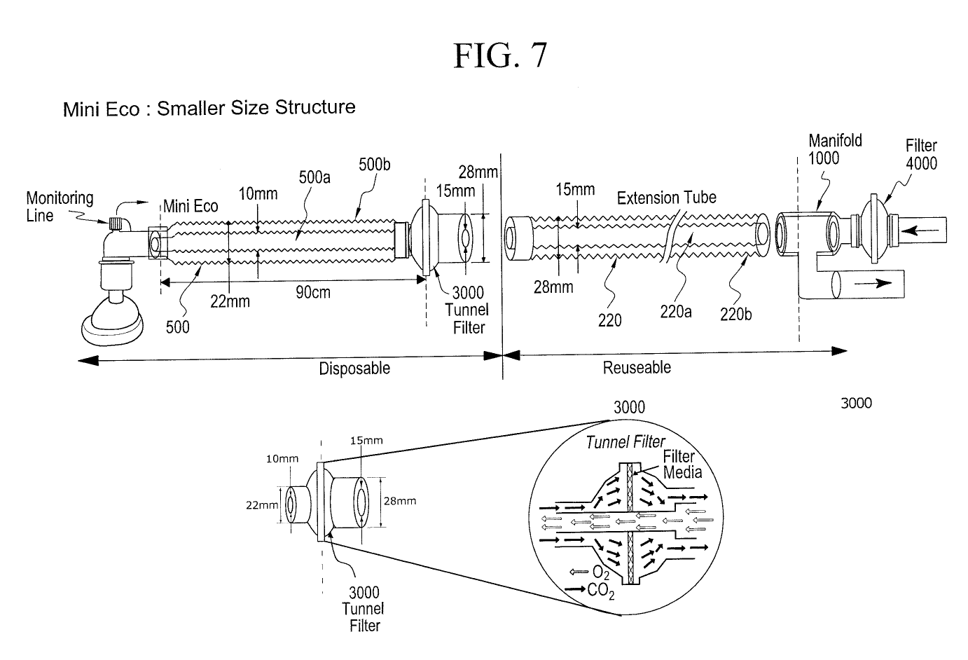

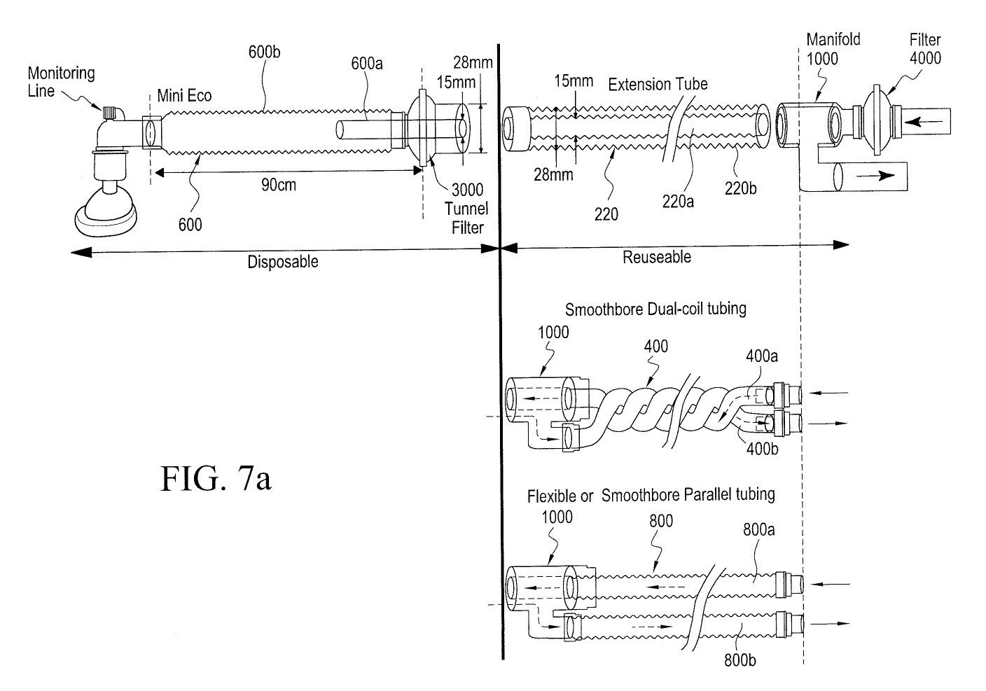

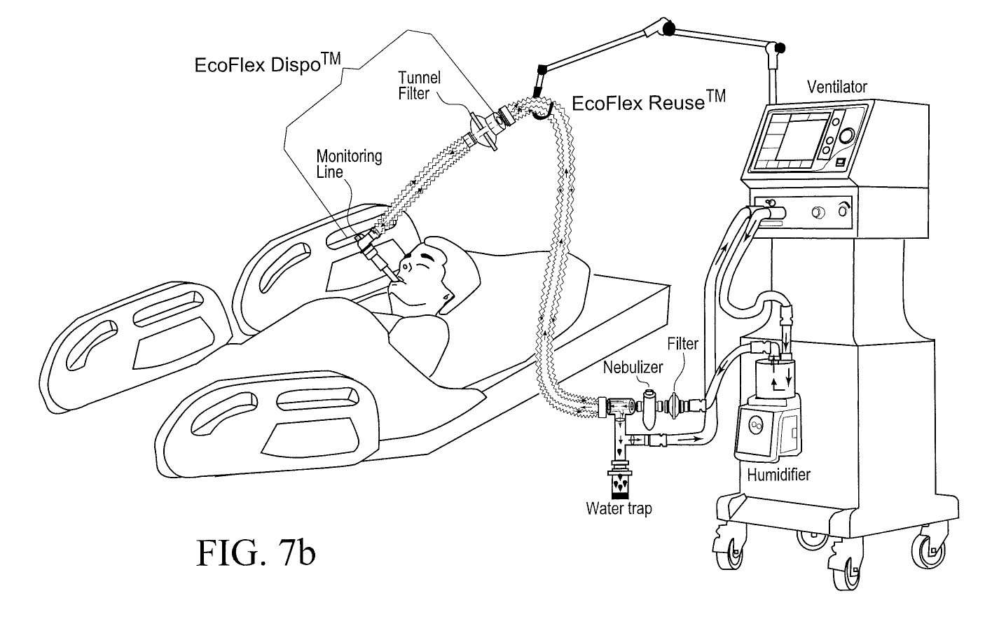

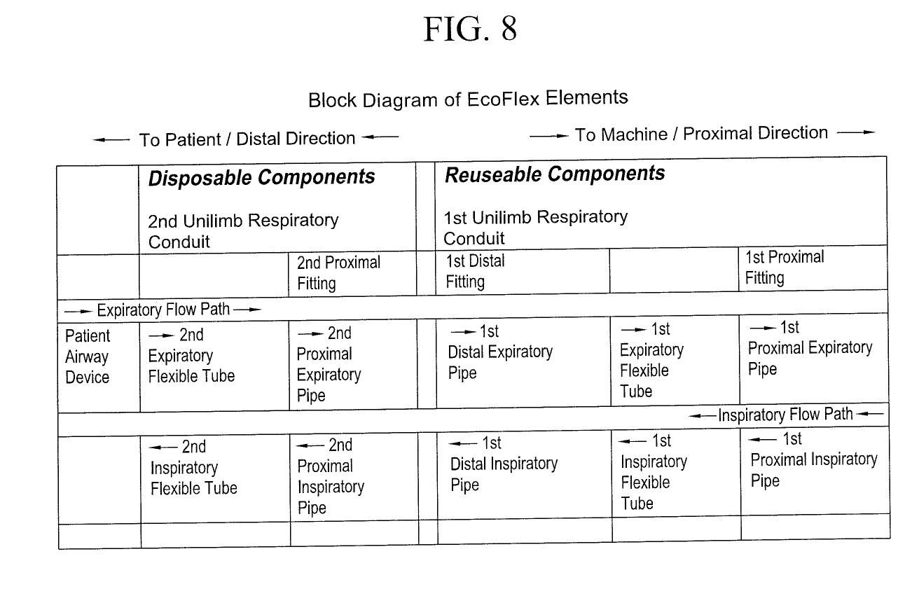

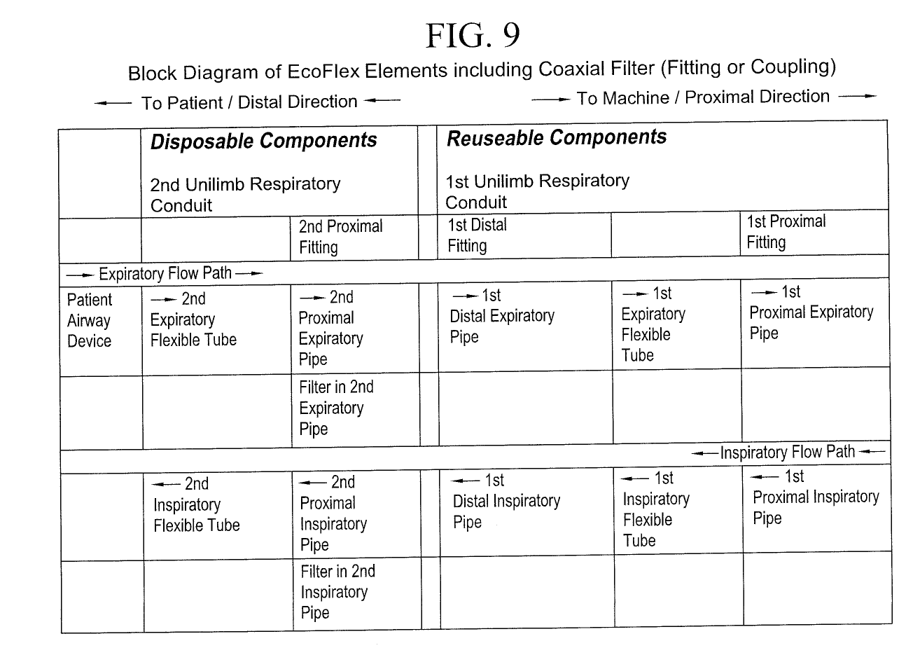

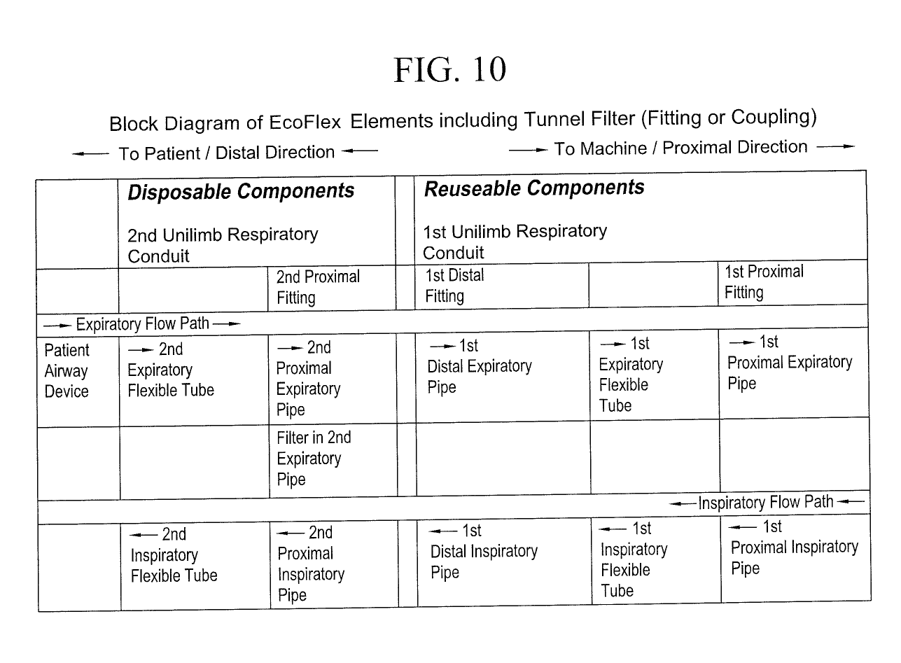

Beneath the Flex2 Circuit in FIG. 6 is illustrated a new system of the present invention, comprising a first disposable section 210, referred to as the EcoFlex Dispo™, comprising an inspiratory conduit 210a and expiratory conduit 210b, and a second reusable section 220, referred to as the EcoFlex Reuse™. As earlier described in general, connectors for flexible tubes, such as 210A, hold the flexible tubing so that the distal end of the flexible tube(s) connects to a patient airway device (e.g., mask, LMA etc.), and in addition can be attachable to an elbow connector 900, and a monitoring line inlet 910. Tubing lengths in inches and centimeters, as well as tubing diameters in millimeters, are as set forth herein and/or correspond to match overall prior art circuits approximate total lengths when the new components of the present invention are assembled into complete circuits for use.

FIG. 6a shows in its upper portion the layout and components of a commercially available breathing circuit known as the Vital Signs Limb-O™, wherein the patient conduit 250 is a single tube that can have a dividing wall 250S to create separate inspiratory 250a and expiratory 250b lumens. The lower portion of FIG. 6a illustrates embodiments of a new system of the present invention wherein the new patient conduit (or EcoFlex Dispo™) 250 can comprise a single tube with a dividing wall 250S to keep inspiratory 250a and expiratory 250b flow paths separate, and an extension tube (or EcoFlex Reuse™) 260 comprising a single tube with a dividing wall 260S to keep inspiratory path 260a and expiratory path 260b separate. In a use that combines a coaxial EcoFlex Dispo and a divided tube EcoFlex Reuse (as in the bottom of FIG. 6a) or a use that combines a divided tube EcoFlex Dispo and coaxial EcoFlex Reuse, a flow director 2700 is used.

FIG. 7 shows in its upper portion another embodiment of a new system of the present inventions, comprising a first disposable section, referred to as the Mini Eco 500, and a second reusable section 220, which is the same as the EcoFlex Reuse™ in FIG. 6. In an embodiment shown in FIGS. 7 and 7a, the diameters of the disposable section 500 include an inner tube of 10 mm and an outer tube of 22 mm, which are smaller than the diameters of the reusable section 220. Below the system diagram is a cross-sectional view of a new Tunnel Filter 3000 of the present invention. Thus, the Mini Eco has tubing of smaller diameter than the tubing of the EcoFlex Reuse™ portion. In an embodiment, the rigid inner and outer pipes that form the proximal and distal ends of the Tunnel filter are sized so that the proximal end of the inspiratory (e.g., inner) pipe fits to a matching tube or pipe having a diameter for example of 15 mm, while the proximal end of the expiratory (e.g., outer) pipe fits to a matching tube or pipe having for example a diameter of 28 mm. Hence, the Tunnel filter can act as a reducing fitting or coupling for distal and proximal components in a circuit without causing significant flow resistance while providing a smaller filter to protect the machine providing inspiratory gases, etc. If desired, a conventional single filter 4000 can be connected to the inspiratory end of the Manifold 1000.

FIG. 7b shows an embodiment of a system for use in patients who require a humidifier and/or a nebulizer (for example, in patients requiring long ventilation, e.g., ICU patients).

FIG. 8 is a block diagram of a new system of the present inventions, having a first disposable section with its components shown under the heading Disposable Components, and a second reusuable section with its components shown under the heading Reusable Components circuit.

FIG. 9 is a block diagram of the system shown in FIG. 8, with the addition of coaxial filter elements into the inspiratory and expiratory flow paths of the 2^{nd} proximal fitting (or coupling) of the disposable 2^{nd} unilimb respiratory conduit.

FIG. 10 is a block diagram of the system shown in FIG. 8, with the addition of a Tunnel filter to the 2^{nd} proximal fitting (coupling). The Tunnel filter includes a filter element in the expiratory path created in the 2^{nd} proximal fitting expiratory pipe (which may have an enlarged cross sectional area to reduce flow resistance caused by the filter medium), while the inspiratory pipes in the 2^{nd} proximal fitting have no filter.

### MORE DETAILED DESCRIPTION OF THE INVENTIONS

### Resuscitators/Oxygenators Having Distal Oxygen Inlets

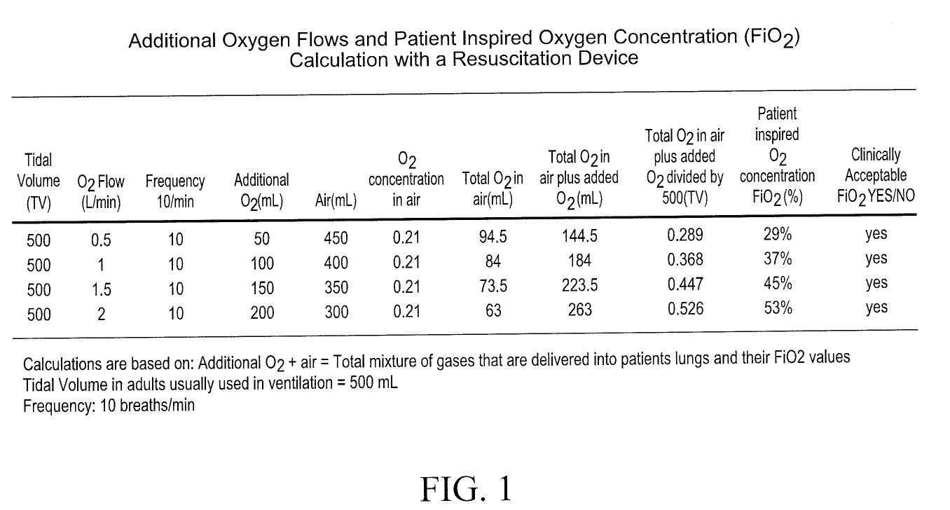

Instead of diluting and wasting O2 with high flows as in the prior art, it was surprising that modifying and relocating the O2 inlet to the distal end of the pump (“Bag”) in a resuscitator, and directing the O2 inflow to the patient airway directly via a breathing conduit (breathing circuit or tube) could achieve significant oxygen savings and/or efficiency as well as ergonomics, allowing more efficient usage of the pump (Bag). In addition, there are tremendous convenience advantages provided by the various configurations of the new resuscitator disclosed herein (FIGS. 3a-1 through 3b-3).

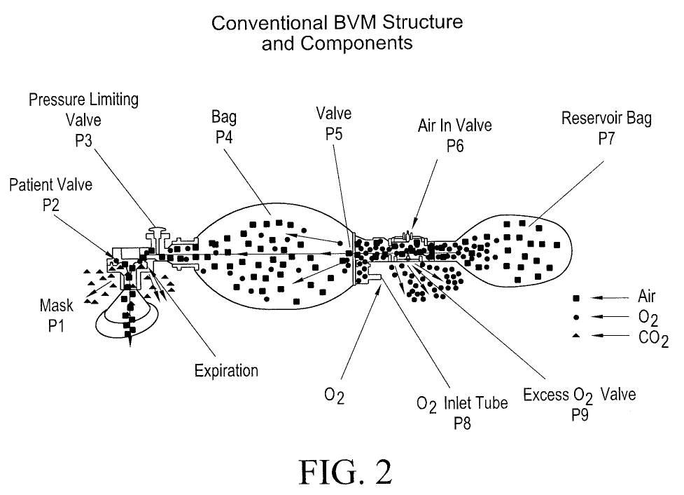

In contrast to the present inventions, the prior art resuscitation devices (BVMs) provide a “reservoir” P7 (bag or long tube) wherein the continuously inflowing O2 is delivered (see the components and configuration in FIG. 2). As shown in FIG. 2, the conventional BVM provides many components and the structure is complex. It requires at least 5 valves, some of them at the distal end of the Bag and the others at the proximal end of the Bag. Furthermore, it is believed that the incoming O2 is diluted in the process of going into the reservoir and the self inflating Bag (by mixing of air and O2), thus requiring high O2 flows to provide high FiO2 so that high O2 flow (≧12 to 15 LPM) is the standard. Unfortunately, with the prior art, most of the inflowing O2 is released out of the system and wasted.

In the present invention, the O2 inflow 5 is delivered directly to the patient's airway without making a detour to the reservoir. In fact, the present invention does not provide a “reservoir”. With the present invention, the inflowing O2 is directed to the patient airway via a breathing conduit 2. This is similar to using the principles of a mechanical ventilator or anesthesia machine wherein the gases are delivered via a breathing circuit. The pump 8 (“Bag”), incorporating a pressure limiting valve 6, can be safely used with low O2 flow (<1 to 2 LPM). Moreover, high O2 inflow is not necessary to achieve clinically acceptable FiO2 as shown by Table 1. Therefore, low O2 flow would be sufficient to achieve clinically satisfactory FiO2; O2 utilization is significantly more efficient compared to the prior art.

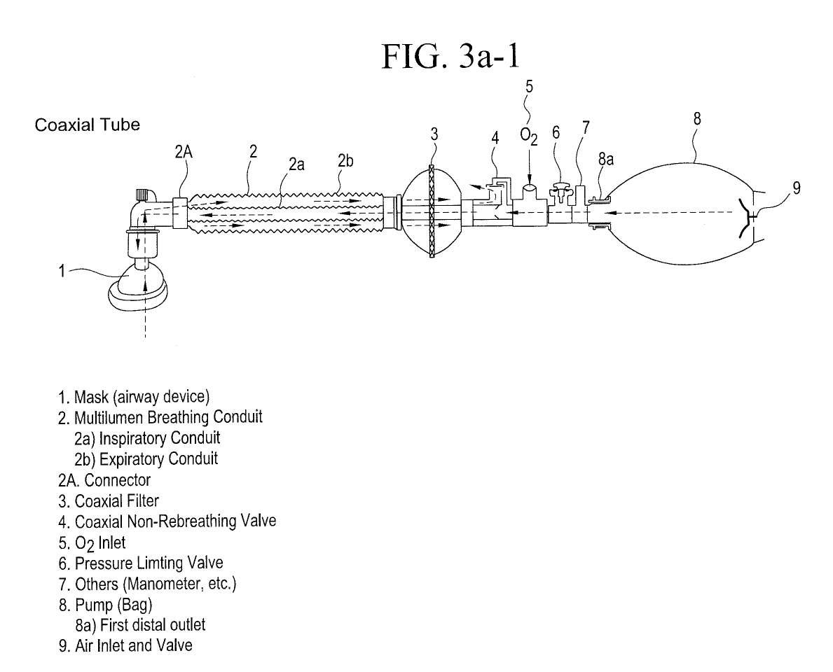

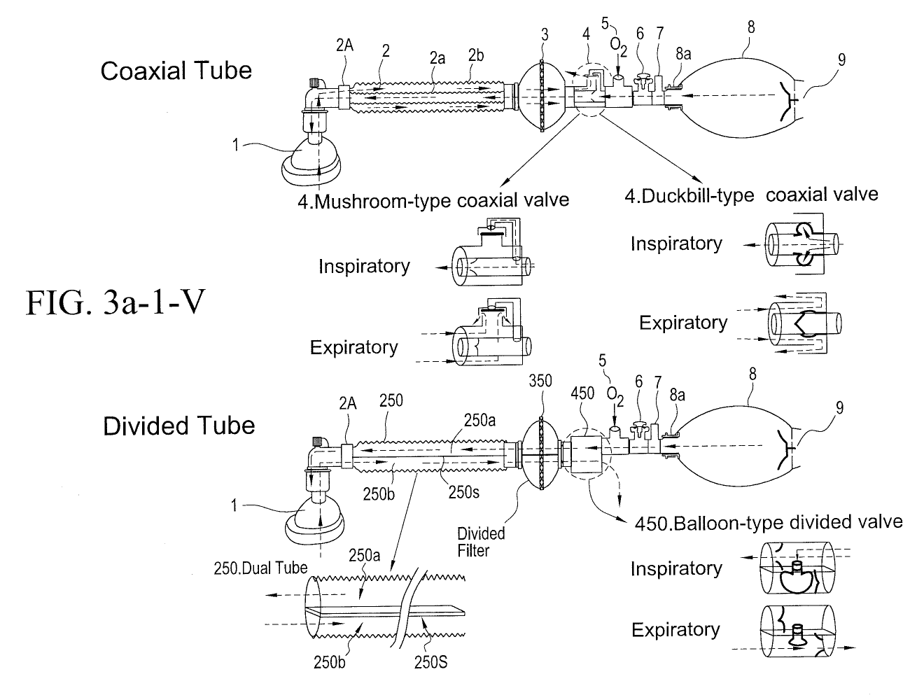

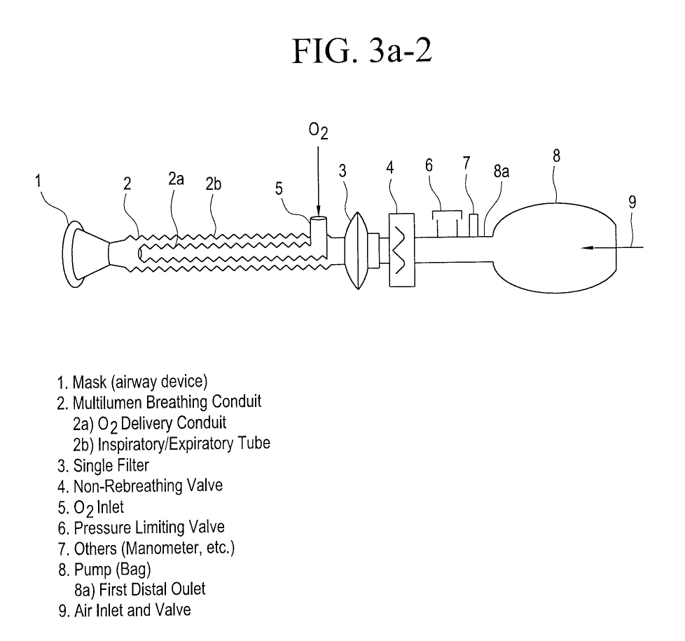

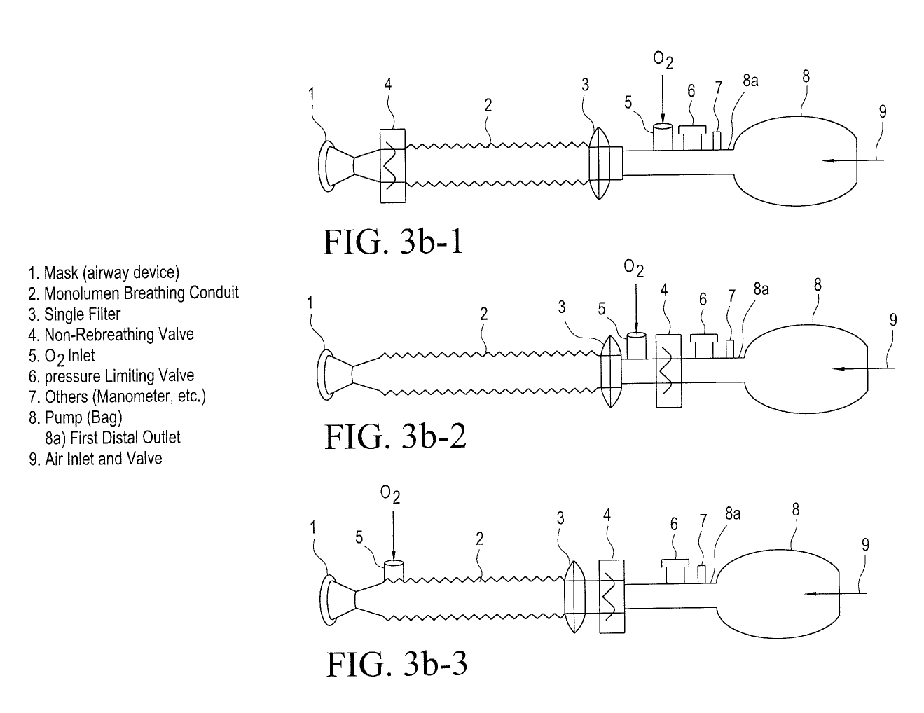

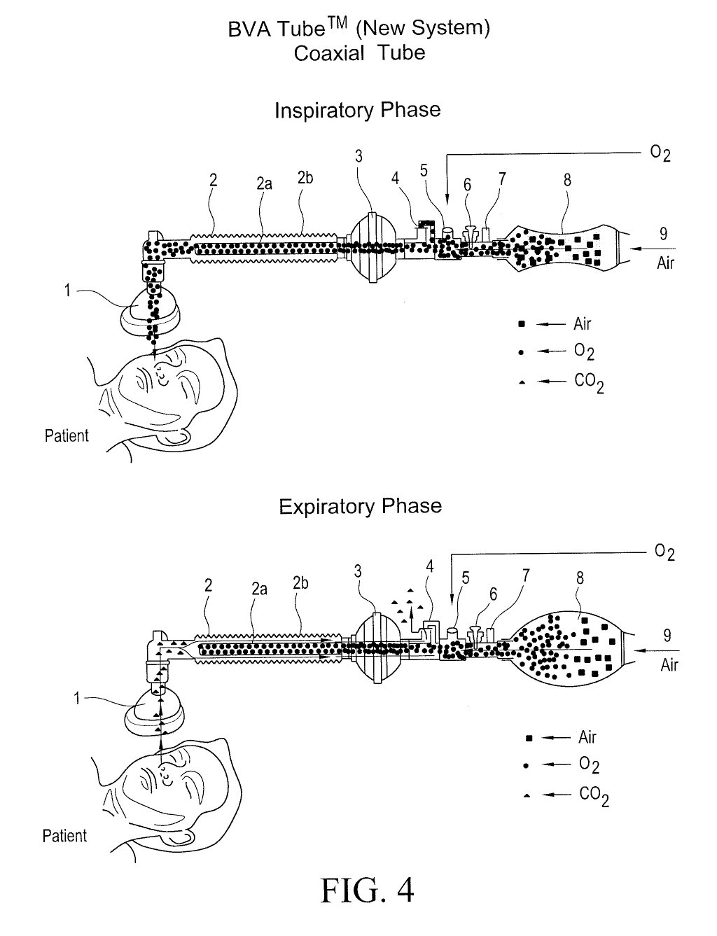

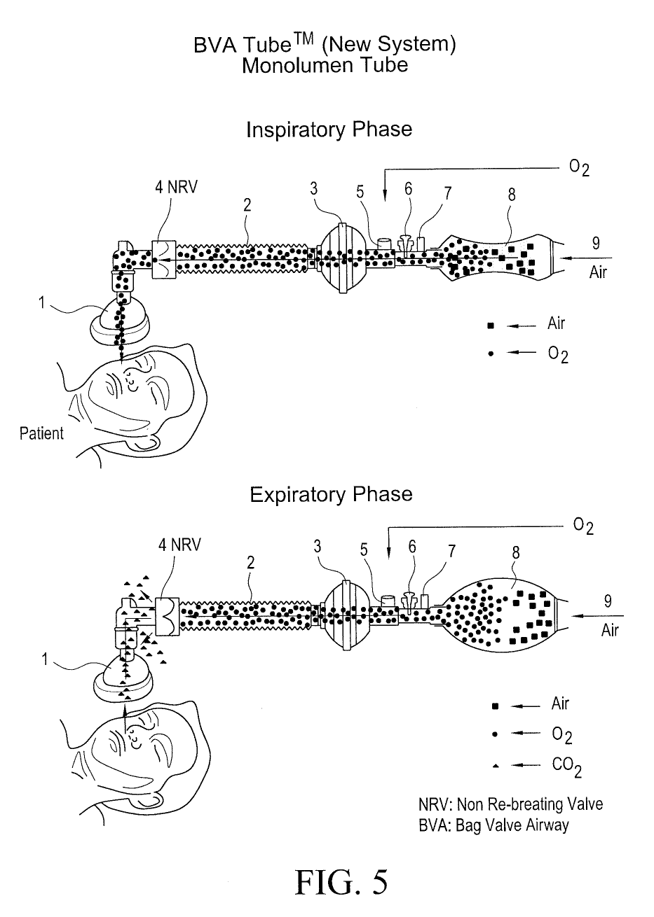

In the event that excessive high flows are inadvertently used, in an embodiment an additional safety measure is a pressure limiting valve, so that barotrauma is avoided. To better appreciate the mechanics or function of the present invention, refer also to FIGS. 3 to 5.

During the inspiratory phase (Bag compression or squeezing phase), the following occur simultaneously:

(A) increased pressure in the Bag causes closing of the Bag refill valve (or air in valve) 9 and closing of the exhalation port of the NRV or patient valve),
(B) the NRV valve or patient valve 4 opens, and
(C) O2 in the breathing conduit 2 and the content of the Bag 8 (O2+air) is pushed directly into the patient's airway via a breathing conduit 2 through the filter 3 and a mask 1 or other airway device (e.g., laryngeal mask (LMA), endotracheal tube (ETT), laryngeal tube (LT), nasal tube (NT)).

During the expiratory phase (Bag decompression, self-inflating Bag or releasing phase), the following occur simultaneously:

(X) air pressure valve 9 opens,
(Y) the NRV valve 4 closes to creates negative pressure and air is drawn into the Bag 8 very quickly (the Bag self inflates instantaneously), and
(Z) the exhalation port of the NRV valve 4 opens and the patient's exhaled gases are released out of the system.

Concurrently with the above, O2 is continuously inflowing into the O2 inlet 5 at the proximal end of the breathing conduit 2. During the expiratory phase, the continuously inflowing O2 is drawn into Bag 8 due to the negative pressure caused by the Bag's self-inflating action; the O2 mixes with the air drawn in from the surrounding atmosphere, and such mixed gases are directly delivered (pushed in) into the patient's airway (oropharyngeal or nasopharyngeal pathway) at the next inspiratory or Bag's squeezing phase. Most importantly, the low flow O2 is not released out of the system but is efficiently used. Thus, there is no need to use high O2 flows of 10 to 15 LPM recommended for prior art resuscitators to achieve adequate FiO2.

In contrast, as shown in FIG. 2, the prior art BVMs and their modifications provide an additional element or component (reservoir P7) wherein the O2 flows into the reservoir P7. Due to having a separate compartment from the self inflating Bag, the O2 inflow does not connect directly to the patient's airway but rather is diverted to the reservoir P7 (i.e., makes a detour to the reservoir before the reservoir's content is drawn into the self inflating Bag P4 and the contents of Bag P4 are delivered to the patient when the Bag is compressed). Thus, although O2 is continuously flowing from the O2 source, with the Prior Art system O2 is delivered indirectly and intermittently to the patient. Consequently, despite high O2 flow, most of the O2 mixes with and is diluted by a large volume of air pulled in from the inlet P6 and/or is released out of the resuscitator from outlet P9 whereby the majority of O2 supplied is vented out and wasted.

BVM ventilation can be and often is life-saving, but the technique presents many problems; achieving an adequate seal between the patient's face and the mask is one of the more challenging components of this procedure, which is mostly due to the configuration of the device (i.e., the bulky Bag proximity to the facial area makes it difficult to keep a good seal of the mask to the patient's face while simultaneously pumping the Bag sufficiently and using a proper inhalation/exhalation sequence to ensure that the patient receives adequate air and 02). Thus, a tight mask seal and adequate compression of the Bag is difficult to achieve with Prior Art devices.

With the present invention, the breathing conduit 2 provides space (distance) between the patient's face and the self inflating Bag 8, which provides many other benefits, such as not obstructing the facial area with the bulky Bag 8, providing good sealing of the mask to the face, nose and mouse, easier bag operation, and delivery of adequate tidal volume by full compression of the Bag.

The use of a multilumen breathing conduit (e.g., coaxial breathing conduit 2, EcoFlex Dispo™210) allows placement of the valves, O2 inlet connector and other components at the proximal end of the breathing conduit, away from the face, which in turn provides ample clearance at the mask connection and helps the rescuer to place the mask with a good seal to the patient's face and airway, provide sufficient compression of the Bag to achieve adequate tidal volume, and avoid hypoventilation. To better appreciate the mechanics and function of the device as well as methods or systems utilizing same refer also to FIGS. 3 to 5.

A modification of the prior art BVM includes the Bag with a single tube between the self inflatable Bag and the mask (e.g., the Laerdal Silicon Resuscitator [LSR] by Laerdal Co., Norway). However, O2 does not flow directly into the patient's airway, rather the high O2 inflow detours to the reservoir and wastes O2 as described previously. In addition, it requires sterilizing and/or disposing the entire device after each use.

In conclusion, prior art BVMs present many disadvantages and problems. These problems and disadvantages can be overcome by new systems of the present inventions, which will be referred to as the Bag+Valve+Airway (“BVA”) Device+Breathing Tube (“BVATube”) (“BiVaTube”) or “F-Bag”.

### The “BVATube”

BVA Tube Elements (Components or Parts) comprise those listed below—Please refer to FIGS. 3 to 5 (although certain parts are not specifically shown therein).

(1) Airway device or Patient Airway Device (which can be selected from a Mask, endotracheal tube (ET), laryngeal mask (LMA), laryngeal tube (LT), nasal tube (NT), inter alia).
(2) Breathing conduit (e.g., mono lumen conduit or multilumen unilimb conduit [e.g., coaxial tubing or circuits: F2/F3] EcoFlex™ 210), which allows oxygen delivery directly to the patient's airway. Note that a multilumen patient airway conduit can be a single conduit with a dividing wall for inspiratory and expiratory flows and have corresponding distal and/or proximal fittings (or couplings) to engage corresponding fittings (couplings), respectively.
(2.1) Elbow (connector between breathing conduit and the airway device preferably with sampling line port). Note that certain kit components for making and using breathing circuits are standard for prior art breathing circuits, and such standard components are included in kits of the present invention, even if not specifically stated herein. For example, sampling lines, breathing bags, HMEs, capnometers, masks, laryngeal tubes, nebulizers and other components used in prior art assisted ventilation and anesthesia, and which practitioners include in a circuit carrying gases to and from a patient, are included in a system, method, circuit and/or kit of the present invention. Such additional components may vary depending on the patient and the procedure, and methods, systems, circuits and kits containing such additional components that are included to form and in the use of systems, methods, devices, circuits and kits of the present invention are included as part of the inventions. A kit, circuit or part of a circuit, or a system with such additional components, whether included in a single kit or order or acquired separately, is thus an embodiment of the present inventions.
(2.1.1) Sampling line (monitoring line).
(3) Filter (including multi-lumen filter e.g., coaxial filter).
(4) Valve (“NRV” Non-rebreathing valve).
(4.1) If a multi-lumen conduit, such as a tube with a dividing wall or a septum (e.g., Limb-O™ by Vital Signs) or a coaxial tube, is used, then a multi-lumen valve is used (e.g., coaxial valve including inspiratory and expiratory valves connecting to the breathing conduit. The valve can be e.g., a mushroom type valve, balloon type valve,duckbill type valve, such as the ones shown in FIG. 3a-1-V).
(5) Oxygen inlet port and/or connector.
(6) Pressure limiting valve (PLV).
(7) Other interface devices e.g., manometer, PEEP valve etc.
(8) Self-inflating bag (“Bag”) is simpler than conventional BVMs or resuscitators as it comprises lesser elements.
(9) Air inlet valve and air in orifices.

### Disadvantages of Prior Art Resuscitator, Known as Bag Valve Mask (BVM)

Prior art BVMs comprise a mask, a non-rebreathing valve, a pressure limiting valve, a self-inflating bag, an oxygen reservoir, a plastic bag or tube including valves, etc. (see FIG. 2). Disadvantages are listed below.

1. Obstruction of facial area.
1.1. Requires at least two (2) people to operate properly.
1.2. Bag is near or close to the patient's face.
1.3. Big bulky bag is difficult to compress and decompress near the patient's face while holding the mask in place.
1.4. Leakage—limits amount of air coming to the patient because of the above problem.
1.5. Obstructs the area around the patient particularly when giving CPR.
1.6. Limits visualization of the chest area, whether the patient is inhaling or exhaling.
   2. Inefficient oxygen utilization.
2.1. High flows of O2 (requires 12 to 15 LPM or more).
   3. No cross infection control (no filtration).
3.1. The whole device needs to be (a) sterilized or (b) disposed of, which:
   3.1.1. Adds cost,
   3.1.2. Adds storage space, and
      Is environmentally unfriendly.
         4, Requires many valves (See FIG. 2).
4.1. Patient valve P2.
4.2. Pressure limiting valve P3.
4.3. Valve P5 (proximal end of the Bag allows inflow of air and O2 to the Bag P4).
4.4. Air in valve P6 (between the Bag and the reservoir).
4.5. Excess oxygen valve P9.

BVA TUBE™ (BIVA TUBE™ OR F-BAG™) IMPROVES AND OVERCOMES ALL THE BVM DISADVANTAGES MENTIONED ABOVE SIMULTANEOUSLY The BVA Tube™ combines a simpler self-inflating bag (“Bag”) and a breathing conduit (“Tube”), for example, a monolumen single flexi tube (accordion-like tube) or a F2 or F3 unilimb coaxial tube with a filter as disclosed for example in U.S. Pat. Nos. 5,983,896 and 7,261,105 respectively. Although we use the term “Tube” for brevity, “Tube” includes all types of breathing conduits (e.g., King F2™ breathing circuits sold by Ambu/King, Limb-O™ circuits by BD/Vital Signs, etc.). With the BVA Tube, all the valves and interface devices are located away from the patient's face and oxygen is provided at a location distal to the Bag outlet in the most efficient manner.

In a preferred embodiment, the breathing conduit (“Tube”) is provided with a filter wherein the filter connects with the NRV and can be attachable/detachable at such junctions; however, the Tube with all the other interface components (e.g., valves, manometer etc.) can be connected to the Bag without the filter. In the latter case, all the components would be sterilized or disposed of.

### BVA Tube™ Advantages

### 1. Avoids Obstruction Near the Face.

1.1. All the valves, O2 supply inlet or connector, interface devices (e.g. PEEP valve, manometer) are located at the proximal end of the breathing conduit (Tube), and distal to the self-inflating bag (Bag); this configuration avoids cluttering and bulkiness at the patient's face, i.e., it clears the space around the facial area.
1.2. Because the breathing conduit (Tube) provides distance between the face and the Bag, placing the mask properly (to maintain an air tight seal) is much easier, which avoids leakage due to improper placement of mask, thus providing better ventilation.
1.3. Facilitates giving CPR because ample space is provided about the patient.
1.4. Rescuers and caregivers can better visualize chest movement (inhalation and exhalation).
1.5. It is easier to compress and decompress the Bag as it is distanced from the patient's face by the breathing conduit.
1.6. The BVA TUBE™ can be used by one person while providing more accurate control of patient ventilation than prior art resuscitators.
1.7. Oxygen is delivered directly into the inspiratory Tube and to a patient.

### 2. Oxygen Utilization Efficiency is Greatly Improved.

2.1. Location of the oxygen delivery inlet at the distal end of the Bag and proximal end of the Tube stores the low flow inflowing oxygen in the Tube and distal of the Bag, instead of diluting or releasing O2 out of the system.
2.2. Users can lower the oxygen delivery flows to 1-2 LPM while maintaining clinically satisfactory FiO2.
2.3. The new Resuscitator/oxygenator of the present invention can use a smaller oxygen tank.
   2.3.1. The Resuscitator/oxygenator enables using oxygen in an oxygen tank more efficiently and for longer time.
   2.3.2. The new Resuscitator facilitates surgeries and emergency treatments in remote areas where modern hospital facilities are limited and/or large oxygen supplies are limited or impractical.
   2.3.3. The new Resuscitator is particularly useful while transporting a patient by ambulance, or air, e.g., helicopter etc.
The New Resuscitator Avoids Cross Contamination and minimizes risks to health practitioners (e.g., paramedics) due to breathing gases expired by the patient.
2.4. A filter located at the proximal end of the Tube prevents contamination of the Bag, O2 connector, valves and interface devices.
2.5. There is no need to dispose of the whole Bag and the valves, which are expensive components, which:
   2.5.1. saves money,
   2.5.2. leads to less parts to dispose of, and is ecologically friendly,
   2.5.3. saves storage space, and
   2.5.4. saves shipping costs.
2.6. The only new Resuscitator part that one needs to dispose of is the Tube and the filter (e.g., Filtered Tube, EcoFlex Dispo™).
   2.6.1. This is more environmentally friendly.
2.7. The new Resuscitator requires fewer valves than the conventional BVM system.
   2.7.1. Reduced number of valves reduces the possibility of valve malfunction (as well as saving on the cost of extra valves used in the prior art).
2.8. The new Resuscitator does not require a reservoir (bag or tube).

### 3. BVA Resuscitator Operation and Explanation.

3.1. In an emergency situation,
3.1.1. A patient receives air (oxygen 21% concentration) via the Bag as a first step.
3.1.2. Medical personnel hook the oxygen tank to the BVATube™ inlet/connector 5.
3.1.3. Oxygen is provided to continuously flow into conduit 2 to the mask 1 and to the patient.
3.1.4. During the inspiratory phase, by squeezing the Bag, conduit 2 serves as a tube to deliver the patient airway device oxygen coming from oxygen inlet 5 plus gas content from the Bag 8 (i.e., O2+air)
3.1.5. Oxygen is directly delivered to the patient's airway via the distal end of the breathing conduit 2.
3.1.6. At the expiratory phase, the NRV 4 causes release of exhaled gases from the system. When a multilumen conduit (FIG. 3a-1) and valve are used, initially, during the expiratory phase the coaxial valve 4 inspiratory valve closes the pathway to inspiratory conduit 2a and releases expiratory gases out of the system via expiratory conduit 2b and the expiratory path in coaxial valve 4, while the continuously inflowing O2 will be initially delivered distal of Bag 8 and into the distal end of Bag 8.
3.1.7. At the inspiratory phase, e.g., when the Bag is squeezed, positive pressure from the bag causes the inspiratory valve to open; the incoming O2 from O2 inlet 5 will go directly to the inspiratory conduit 2a while the Bag's content, which has an enriched O2 concentration from mixing O2 with air from inlet 9, will also be forced through inspiratory conduit 2a to the patient.
3.1.8. Thus, the patient receives oxygen rich (e.g., 30-50%) inspiratory gases.
3.1.9. This can be safely accomplished with low O2 flows (1-2 LPM), and without waste because the new Resuscitator fully utilizes the O2 flow to the system
3.1.10. The result is that all the “low flow” oxygen is delivered to the patient who receives the maximum amount of high concentration oxygen FiO2, which is in contrast to the high flows (12-15 LPM) required by the conventional prior art devices and systems.
3.1.11. The respiratory cycle is effected by synchronizing the physiological pattern of breathing (inspiratory phase and expiratory phase on 1:2 ratio approximately) and by providing the inflowing O2 directly to the patient via the breathing conduit (which can be monolumen, or multilumen unilimb circuit (e.g., EcoFlex Dispo).
3.1.12. When the crucial, emergent phase is past and the situation becomes stable, the mask can be replaced, for example with a laryngeal mask (LMA) or endotracheal tube (ET), which provides oxygenation in a more favorable manner.
3.1.13. Medical personnel do not need to hand hold the mask.
3.1.14. Mask leakage is reduced in comparison to prior art systems.
3.1.15. Once the airway device, for example a LMA is in place, oxygenation can be provided with the Bag and low flow O2 for a very long time and with a small O2 tank. The system of the present inventions greatly multiplies the usage time of a fixed oxygen supply (e.g., oxygen tank). For example, an O2 tank containing 660 L supplied @ 0.5 to 1 LPM could provide from 10 to 20 hours of use).
3.1.16. The BVA system can, in some circumstances, expand use from acting as a resuscitator to serving as a simple ventilator to provide ventilation and/or anesthesia (e.g., total intravenous anesthesia while maintaining adequate ventilation in the absence of big, complex and expensive anesthesia machines and/or mechanical ventilators).
3.1.17. In fact, the BVA system may be particularly useful for procedures in outpatient surgical offices, dental offices, diagnostic office (e.g., endoscopy, MRI) in rural areas or countries where resources are limited.
3.1.18. If the system is used for transporting patients, the EcoFlex Dispo™ can be readily connected to the EcoFlex Reuse™ in the ambulance or the hospital (See FIGS. 6 to 7b).

### Breathing Circuits Having Reusable and Disposable Components

The present invention involves a novel ventilation or anesthesia system and method to provide ventilation or anesthesia that has a reusable portion and a disposable portion in the same system or breathing circuit, unlike prior art circuits and systems which required disposing of the entire breathing circuit. After use by a patient, a smaller amount of the breathing circuit (compared to the prior art), together with a disposable filter is disposed of, leading to reduced supply costs and reduced medical wastes, yet improving or maintaining patient safety.

Respiratory patency must be maintained at all times. Preferably the resistance should be less than about 1 cm H2O pressure drop at 10 L/min or about 6 cm H2O pressure drop at 60 L/min. Therefore, a screening test should be done at various conditions and flow rates (e.g., 0.5 L/min to about 60 L/min with various conduit diameter and conduit lengths). The resistance should be within the acceptable ranges (i.e., low resistance) to meet the requirements for spontaneous or assisted ventilation.

In a preferred embodiment, a breathing circuit having substantially minimal flow resistance to spontaneous breathing or assisted ventilation has a smaller portion that is disposable and a larger portion that is reusable than in prior art circuits. The disposable components of the present invention circuits have at least two lumens: one for inspiratory and the other for expiratory pathways. The disposable components of the present invention circuits are particularly small in comparison to prior art circuits of about the same length and the components disposed of are significantly reduced with circuits of the present inventions. Block Diagrams of preferred embodiments of these systems are shown in FIGS. 8 to 10, with illustrations of components in embodiments shown in FIGS. 6 and 7.

In an embodiment, the patient or distal end of a breathing circuit or device has a small, short conduit and/or filter portion that is disposable, referred to as a “distal disposable breathing device” or “distal disposable filter and tube device”, while the proximal or machine side portion is reusable. For the sake of convenience, a distal disposable filter and tube(s) device conforming to a preferred embodiment of the present invention is referred to as the EcoFlex Dispo™ (see left side of FIG. 6, lower figure). In an embodiment, the filter and the tubing are bonded and integrally constructed. The length of the tubing in the distal disposable breathing device is long enough to keep a filter or other device (e.g., HME) connected thereto sufficiently far away from the patient's face so as not to interfere with medical care being provided to the patient, yet short enough to reduce the amount of material that is contaminated by a patient that requires disposal or sterilization. In embodiments of the present inventions, the length of the distal disposable breathing device can be, for example, between about 10 cm and about 90 cm, between about 20 cm and about 60 cm, and between about 30 cm and about 40 cm.

An alternative embodiment of the EcoFlex Dispo™ device includes an adjustable length distal breathing tube (e.g., flexitube), which places a patient airway device in fluid communication with the proximal portion of a circuit via a filter. Preferably, the filter and tube are bonded together to form an integral device. The proximal portion of a breathing circuit that incorporates an EcoFlex Reuse™ may optionally include an adjustable length proximal tube that permits further adjustment of the length in the circuit.

In contrast to the prior art, in a preferred embodiment, a disposable conduit (e.g., EcoFlex Dispo™ in breathing circuit embodiments of the present inventions has at least two lumens (inspiratory and expiratory lumens) that couple with corresponding filter pipes (e.g., FIGS. 6, 6a, 7, 7a and 7b).

In contrast to the prior art, a filter in breathing circuit embodiments of the present inventions is located neither at the distal end or the proximal end of the breathing circuit. The filter in the present inventions is located at a point between the distal and proximal end of the breathing circuit to minimize medical waste while maintaining patient safety and further being effective and practical. A preferred distance between the filter and the distal end of the distal disposable breathing device is between about 10 cm and about 90 cm, more preferably between about 20 cm and about 90 cm. Hence, the filter could be said to be intermediately placed in a breathing circuit.

An intermediate circuit fitting (or coupling) of the present invention permits ready connection and disconnection of the distal disposable filter device (EcoFlex Dispo™) of the present invention to reusable circuit components of the present invention. For example, rigid distal fitting 225 in FIG. 6 and rigid distal fitting 265 in FIG. 6a facilitate mating attachment to the proximal end of the filter or proximal end fitting of the EcoFlex Dispo™. Reusable circuit components (EcoFlex Reuse™) refer to coaxial tubing such as that sold by Ambu/King Systems as Universal Flex2™ Extension tubing, inter alia.

In an embodiment, a distal filter device (i.e., a filter and distal breathing conduit used at the patient side of the breathing system or EcoFlex Dispo™) has a fresh gas flow outlet near to or at the distal terminus of the EcoFlex Dispo™ device, wherein the distal terminus can be connected to a patient airway device (e.g., mask, laryngeal mask, etc).

With respect to the manifold 1000 shown in FIG. 6, it includes a Proximal Terminal, described in prior patents, e.g., U.S. Pat. No. 5,778,872, which permits two independent flows, such as an inspiratory flow in a lumen from an gas inlet on a machine and an expiratory flow in a lumen to a gas outlet on a machine, to be merged into a single or unilimb circuit to provide gases to and exhaust gases from a patient. The Proximal terminal has a distal end that couples or fits to a mating proximal fitting or coupling, such as the Coaxial Filter shown in the upper portion of FIG. 6. The proximal end of the Coaxial Filter has two pipes or tubes of more rigid material, which can engage matching pipes or rigid tubes at the distal end of the proximal terminal. The distal end of the Coaxial Filter has two pipes or tubes of more rigid material, which can engage or be bonded to the proximal ends of flexible tubes that carry gas to and from the mask (or other patient airway device) at the distal end of the circuit. In an embodiment, the distal pipes or tubes of the Proximal terminal can be directly attached or bonded to flexible tubes that carry inspiratory and expiratory gases to a distal fitting that will engage with the proximal end of a proximal fitting (or the Coaxial filter shown in FIG. 6). It should be noted that other configurations of tubing and fittings are envisioned. For example, a proximal terminal and fittings can be made so that it can connect to separate flow pathways, which in turn can connect to a divided tube single limb breathing circuit (e.g., Limb-O™). Likewise, a multilumen and multichamber filter (operatively connectable with inspiratory and expiratory lumens of the breathing conduit) can be provided therewith, (e.g., FIG. 6a, upper right). Various combinations of unilimb components can be used (e.g., FIG. 6a, lower).

In an embodiment, the coaxial filter/fitting or coupling, includes expanded diameter portions between the distal and proximal ends, which permit placement of filter media in corresponding enlarged chambers therein. This permits filtration of inspiratory and expiratory gas flows, which protects the EcoFlex Reuse portion of the circuit in the system. However, an embodiment of the present inventions has filter media provided only in the expiratory lumen.

The absence of the filter media in the inspiratory lumen allows delivering the fresh gas flow with minimum resistance, which may be helpful when the breathing circuit is used in patients with respiratory problems and/or used with a humidifier and/or a nebulizer (for example, in patients requiring long ventilation, e.g., ICU patients). An embodiment is illustrated in FIGS. 7 and 7b wherein the filter in the EcoFlex Dispo is a Tunnel type filter.

With respect to manifold 1000 shown in FIG. 7, the proximal terminal of FIG. 6 is modified to include or to permit the fitting of a single lumen filter 4000 on the proximal end of the inspiratory gas input lumen. Note that the disposable portion of the circuit includes a multilumen filter (capable of connecting with inspiratory and expiratory lumens of the breathing conduit), which can be a Tunnel filter 3000 that is connected at the proximal end of flexible inner and outer tubes. In an embodiment, the Tunnel filter has an expanded diameter portion of the outer pipe between its proximal and distal ends, which forms a filter chamber, while the inner pipe retains the same diameter from its proximal to distal ends, which enables the proximal and distal inner pipe ends to couple with correspondingly sized flexible tubing. However, in an embodiment referred to as the Mini Eco (because less material is used), the diameter of the inner pipe 500a distal end is smaller than the diameter of the proximal end of inner pipe 220a, and the diameter of the outer pipe 500b distal end is smaller than the one of proximal end of outer extension tube 220b.

The Tunnel Filter (fitting/reducing coupling) permits connection to larger diameter inspiratory and expiratory tubing at its proximal end and connection to smaller diameter inspiratory tubing at its distal end. The flow of fresh gases (e.g., oxygen) through the inner lumen does not permit contamination from a patient to reach the Reusable circuit component, while the absence of an inner filter in the Tunnel filter and the shorter length of the Disposable EcoFlex inspiratory and expiratory tubes lack sufficient flow resistance to interfere with respiration and anesthesia techniques; this is despite the smaller diameters of the inner and outer lumens (e.g., 10 mm and 22 mm respectively) that connect to the inspiratory and expiratory lumens (e.g., 15 mm and 28 mm respectively of the inner and outer lumens of the reusable Extension Tube 220). While some preferred part dimensions are mentioned herein, it is to be understood that the dimensions mentioned are exemplary and not limiting. In another embodiment referred to as SuperEco, the disposable tunnel filter 3000 and tube 600, which provides adjustable dead space by axially expanding and contracting the outer tube 600b while the inner tube 600a is of a fixed length. The lower portion of FIG. 7a shows variations of the reusable portion. For example it may comprise parallel dual coil tubing 400 (comprising inspiratory lumen 400a and expiratory lumen 400b) that connect with manifold 1000. In another embodiment, the Reusable portion could comprise flexible or smoothbore tubing 800 comprising inspiratory tubing 800a and expiratory tubing 800b that connect to manifold 1000.

The foregoing inventions have been described with reference to nonlimiting and exemplary embodiments intended to demonstrate the features and benefits of the present inventions, which may be practiced differently than described without departing from the spirit and scope of the invention. For example, the new combination of disposable and reusable parts that form a new breathing circuit, can be applied to form a new resuscitator of the present invention that integrates the breathing circuit (disposable section) with the Bag (pump including valves) to provide new systems and methods for providing resuscitation, oxygenation and assisted ventilation as described earlier in the present application. The present inventions provide great EEEE benefits, e.g, they are Economical, Ecologically friendly, have Expanded uses, and help provide Excellent care, which is due to ergonomics and making it easier to resuscitate, oxygenate and maintain assisted ventilation, provide greater space around the patient's face, greatly improve efficiency in oxygen use which will increase the availability and use of assisted ventilation to remote and in emergent situations.

I claim:

1. A resuscitation device, comprising:
a pump having a proximal end and a distal end, said pump having a first inlet that permits gases to enter said pump but not exit, and said distal end of said pump having a first outlet that permits gases to exit said pump, wherein said pump can be operated to receive and expel gases, a first conduit having a distal end and a proximal end, wherein said proximal end of said first conduit is operatively connected to said first outlet at the distal end of said pump,
a second inlet for receiving gases operatively connected distally of said first outlet of said pump,
said distal end of said first conduit being operatively connectable to a patient airway device, wherein when a patient airway device is present the patient airway device comprises one of the group consisting of a mask, an endotracheal tube, a laryngeal mask, a laryngeal tube, and a nasal tube,
wherein said first conduit can act as a reservoir, and
wherein said pump can receive air from the atmosphere surrounding said device and expel air into said first conduit, and said second inlet can receive oxygen, and
wherein, when oxygen enters said second inlet and said pump is operated to expel air, the oxygen from said second inlet is combined with air expelled from said pump.

2. The resuscitation device of claim 1, further comprising a non-rebreathing valve operatively connected to said first conduit, and
one of the group consisting of:
a filter operatively connected distally of said non-rebreathing valve and to the proximal end of said first conduit,
a filter operatively connected to the proximal end of said first conduit and operatively connected proximally to said non-rebreathing valve, and
a filter operatively connected to the proximal end of said first conduit and said non-rebreathing valve is operatively connected to said distal end of said first conduit.

3. The resuscitation device of claim 1, wherein said pump comprises an elastomeric bag,

4. The resuscitation device of claim 1, further comprising a non-rebreathing valve operatively connected to said first conduit, and
wherein said non-rebreathing valve has an inner valve conduit in fluid communication with said second inlet and said first outlet, and an outer valve conduit that can release gases from said device.

5. The resuscitation device of claim 1, further comprising a non-rebreathing valve operatively connected to said first conduit, and
wherein said non-rebreathing valve has an inspiratory valve conduit in fluid communication with said second inlet and said first outlet, and an expiratory valve conduit that can release gases from said device, wherein at least a portion of said inspiratory valve conduit and said expiratory valve conduit are contained within a single tube, wherein said single tube has a dividing wall dividing said inspiratory valve conduit and said expiratory valve conduit.

6. The resuscitation device of claim 4, wherein said first conduit comprises an inspiratory conduit and an expiratory conduit,
wherein said inspiratory conduit is in fluid communication with said inner valve conduit of said non-rebreathing valve, and said expiratory conduit is in fluid communication with said outer valve conduit.

7. The resuscitation device of claim 5, wherein said first conduit comprises and inspiratory conduit and an expiratory conduit,
wherein said inspiratory conduit is in fluid communication with said inspiratory valve conduit and said expiratory conduit is in fluid communication with said expiratory valve conduit, wherein at least a portion of said first conduit comprises a single tube, wherein said single tube has a dividing wall separating said inspiratory conduit from said expiratory conduit.

8. The resuscitation device of claim 6, further comprising a multilumen filter having an expiratory chamber in fluid communication with said expiratory conduit and an inspiratory chamber in fluid communication with said inspiratory conduit.

9. The resuscitation device of claim 7, further comprising a multilumen filter having an expiratory chamber in fluid communication with said expiratory conduit and an inspiratory chamber in fluid communication with said inspiratory conduit.

10. The resuscitation device of claim 2, wherein said filter is operatively attached proximally to said first conduit, and said filter and said first conduit can be attached to said resuscitation device for use with a first patient and detached therefrom, and
wherein said first conduit and other components operatively attached distally to said filter can be thereby attached for use to said resuscitation device and detached after use for continued use for the first patient, disposed of, or sterilized, and
wherein components of said resuscitation device operatively attached proximally to said filter can be reused.

11. The resuscitation device of claim 2, further comprising a pressure limiting valve operatively connected to said first outlet at the distal end of said pump.

12. A system for providing assisted ventilation and anesthesia that passes inspiratory gases from an inspiratory gas outlet via a first flow path to a patient airway device and carries exhaust gases from a patient airway device via a second flow path to an expiratory gas outlet, comprising:
a first unilimb respiratory conduit, said first unilimb respiratory conduit capable of being used for more than one procedure or for more than one patient, comprising:
a first expiratory flexible tube, and a first inspiratory flexible tube, said first expiratory flexible tube and first inspiratory flexible tube each having a proximal end and a distal end;
a first proximal fitting, said first proximal fitting located at said proximal end of said first unilimb respiratory conduit, and comprising a first proximal inspiratory pipe and a first proximal expiratory pipe, said first proximal expiratory pipe and first proximal inspiratory pipe each having a proximal end and a distal end,
wherein said distal end of said first proximal inspiratory pipe is operably connected to said proximal end of said first inspiratory flexible tube and said distal end of said first proximal expiratory pipe is operably connected to said proximal end of said first expiratory flexible tube,
said first proximal fitting being operably connectable to an assisted ventilation machine so that the inspiratory gas outlet thereof is in fluid communication with said first inspiratory flexible tube and said first expiratory flexible tube is in simultaneous fluid communication to the expiratory gas port of the assisted ventilation machine, and
wherein said distal end of said first unilimb respiratory conduit comprises a first distal fitting that comprises a first distal inspiratory pipe and a first distal expiratory pipe, said first distal expiratory pipe and first distal inspiratory pipe each having a proximal end and a distal end,
wherein said proximal end of said first distal inspiratory pipe is operably connected to said distal end of said first inspiratory flexible tube and said proximal end of said first distal expiratory pipe is operably connected to said distal end of said first expiratory flexible tube,
said system further comprising, a second unilimb respiratory conduit,
said second unilimb respiratory conduit being attachable to said first unilimb respiratory conduit for use in providing assisted ventilation or anesthesia to a patient and detachable therefrom for disposal or sterilization, said second unilimb respiratory conduit comprising a second proximal fitting that operatively connects to said first distal fitting, a second inspiratory flexible tube, and a second expiratory flexible tube,
wherein said second proximal fitting has a second proximal inspiratory pipe that operatively connects with said first distal inspiratory pipe to be in fluid communication therewith to form an inspiratory lumen from said proximal end of said first unilimb respiratory conduit to said distal end of said second unilimb respiratory conduit,
said second proximal fitting having a second proximal expiratory pipe that operatively connects with said first distal expiratory pipe to be in fluid communication therewith to form an expiratory lumen from said distal end of said second unilimb respiratory conduit to said proximal end of said first unilimb respiratory conduit,
wherein said inspiratory lumen can independently carry inspiratory gases to a patient from an inspiratory gas outlet while said expiratory lumen can carry expiratory gases from a patient to an expiratory gas outlet,
wherein said second unilimb respiratory conduit can be operably connected at its distal end to a patient airway device, whereby when said unilimb conduits are connected to a mammal via a patient airway device, a user may connect said first proximal fitting to an assisted ventilation machine in order to provide inspiratory gases and exhaust expiratory gases from a mammal, and
wherein, when said second unilimb respiratory conduit further comprises a filter operatively connected at the proximal end thereof, or when said second proximal fitting comprises a filter in at least said second proximal expiratory pipe, a user may disconnect said second unilimb respiratory conduit after use for disposal or sterilization, while said first unilimb respiratory conduit may be reused.

13. The system of claim 12, wherein said second proximal fitting has a first filter chamber portion in said second proximal expiratory pipe that has a greater cross-sectional area than the cross-sectional area of the remainder of said second proximal expiratory pipe, and a second filter chamber portion in said second proximal inspiratory pipe that has a greater cross-sectional area than the cross-sectional area of the remainder of said second proximal inspiratory pipe, and filters are situated in each of said first and second filter chamber portions for filtering inspiratory and expiratory gases.

14. The system of claim 12, wherein said first inspiratory flexible tube and said first expiratory flexible tube are formed by a dividing wall in a single conduit, or at least a portion of said first inspiratory flexible tube is inside of said first expiratory flexible tube, or said first inspiratory flexible tube is located in a different conduit from said first expiratory tube.

15. The system of claim 14, wherein said pipes in said fittings are matched to the relative orientation of said flexible tubes in order to create separate inspiratory and expiratory flow paths, said pipes having sufficient rigidity to permit interconnection to matching fittings.

16. The system of claim 12, wherein said second proximal expiratory pipe of said second proximal fitting has a first filter chamber portion that has a greater cross-sectional area than the cross-sectional area of the proximal portion of said second proximal expiratory pipe, said second proximal expiratory pipe having a diameter and shape at its proximal end matched to mate with said first distal fitting expiratory pipe so as to form a portion of an expiratory flow path when connected thereto, and a filter is contained within said second proximal expiratory pipe.

17. The system of claim 16, wherein the distal end portion of said second proximal expiratory pipe of said second proximal fitting has a diameter that is smaller than the diameter of the proximal end of said second proximal expiratory pipe, and said distal end of said second proximal inspiratory pipe has a smaller diameter than the proximal end of said second proximal inspiratory pipe,
wherein said second inspiratory flexible tube and said second expiratory flexible tubes have diameters that are smaller, respectively, than said first inspiratory and first expiratory flexible tubes.

18. The system of claim 17, wherein said first expiratory flexible tube has a diameter of about 28 mm, and said first inspiratory flexible tube has a diameter of about 15 mm.

19. The system of claim 17, wherein said second expiratory flexible tube has a diameter of about 22 mm, and said second inspiratory flexible tube has a diameter of about 10 mm.

20. The system of claim 13, wherein said proximal and distal fitting inspiratory pipes have fitting portions at each end thereof having diameters that correspond to the inspiratory flexible tube to which they are attached, and said proximal and distal fitting expiratory pipes have fitting portions at each end thereof having diameters that correspond to the expiratory flexible tube to which they are attached.

21. The system of claim 16, wherein said second proximal inspiratory pipe of said second proximal fitting does not include a filter.

22. The system of claim 12, wherein said second unilimb respiratory conduit further comprises a filter operatively connected at the proximal end thereof, or said second proximal fitting comprises a filter in at least said second proximal expiratory pipe,

23. A kit for use in constructing the system of claim 12, further comprising at least one of the group consisting of:
a patient airway device,
a sampling line,
a heat and moisture exchange device,
a capnometer,
a breathing bag,
a reservoir,
an elbow, and
a nebulizer.

24. The kit of claim 23, wherein said patient airway device comprises one of the group consisting of a mask, an endotracheal tube, a laryngeal mask, a laryngeal tube, and a nasal tube.

25. A method of providing assisted ventilation or anesthesia to a patient, said method comprises the steps of operatively connecting the second unilimb respiratory conduit of claim 12 to an assisted ventilation machine to enable inspiratory gases from the assisted ventilation machine to be provided to a patient when connected thereto and for exhaust gases from the patient to be exhausted through the assisted ventilation machine.

26. A method of providing resuscitation, oxygen, or assisted ventilation to a patient, comprising the steps of using the device of claim 1 to resuscitate, provide oxygen to, or provide assisted ventilation to a patient.

27. A method of providing resuscitation, oxygen, or assisted ventilation to a patient, comprising the steps of:
using a resuscitation device to resuscitate a patient, provide oxygen thereto, or provide assisted ventilation thereto,
disconnecting a first portion of said resuscitation device from said resuscitation device, and
connecting said first portion to a second device, wherein said second device is operatively connected to an assisted ventilation machine, wherein
said resuscitation device comprises:
a pump having a proximal end and a distal end, said proximal end of said pump having a first inlet that permits gases to enter said pump but not exit, and said distal end of said pump having a first outlet that permits gases to exit said pump, wherein said pump can be operated to receive and expel gases, a first conduit having a distal end and a proximal end, wherein said proximal end of said first conduit is operatively connected to said first outlet at the distal end of said pump,
a second inlet for receiving gases operatively connected distally of said first outlet of said pump,
said distal end of said first conduit being operatively connectable to a patient airway device, wherein when a patient airway device is present the patient airway device comprises one of the group consisting of a mask, an endotracheal tube, a laryngeal mask, a laryngeal tube, and a nasal tube,
a non-rebreathing valve operatively connected to said first conduit, and
a pressure limiting valve operatively connected to said first outlet at the distal end of said pump,
wherein said first conduit can act as a reservoir, and
wherein said pump can receive air from the atmosphere surrounding said device and expel air into said first conduit, and said second inlet can receive oxygen, and wherein, when oxygen enters said second inlet and said pump is operated to expel air, the oxygen from said second inlet is combined with air expelled from said pump, wherein:
said first portion of said resuscitation device that is disconnected in said disconnecting step comprises a proximal end and a distal end, wherein a filter is attached to said proximal end, and wherein said filter and components of said resuscitation device attached distally of said filter from said first portion,
wherein said second device comprises:
a first unilimb respiratory conduit, said first unilimb respiratory conduit capable of being used for more than one procedure or for more than one patient, comprising:
a first expiratory flexible tube, and a first inspiratory flexible tube, said first expiratory flexible tube and first inspiratory flexible tube each having a proximal end and a distal end;
a first proximal fitting, said first proximal fitting located at said proximal end of said first unilimb respiratory conduit, and comprising a first proximal inspiratory pipe and a first proximal expiratory pipe, said first proximal expiratory pipe and first proximal inspiratory pipe each having a proximal end and a distal end,
wherein said distal end of said first proximal inspiratory pipe is operably connected to said proximal end of said first inspiratory flexible tube and said distal end of said first proximal expiratory pipe is operably connected to said proximal end of said first expiratory flexible tube,
said first proximal fitting being operably connectable to an assisted ventilation machine so that the inspiratory gas outlet thereof is in fluid communication with said first inspiratory flexible tube and said first expiratory flexible tube is in simultaneous fluid communication to the exhaust outlet of the assisted ventilation machine, and
wherein said distal end of said first unilimb respiratory conduit comprises a first distal fitting that comprises a first distal inspiratory pipe and a first distal expiratory pipe, said first distal expiratory pipe and first distal inspiratory pipe each having a proximal end and a distal end, wherein said proximal end of said first distal inspiratory pipe is operably connected to said distal end of said first inspiratory flexible tube and said proximal end of said first distal expiratory pipe is operably connected to said distal end of said first expiratory flexible tube,
wherein said connecting step comprises connecting said filter of said first device to said first distal fitting.
